(19) United States
(12) Patent Application Publication
(10) Pub. No.: US 2022/0370504 A1
(43) Pub. Date: Nov. 24, 2022

(54) METHOD FOR THE PRODUCTION OF MÜLLER CELLS AND CELL PRODUCT

(71) Applicant: UCL BUSINESS LTD, London (GB)

(72) Inventors: Gloria Astrid LIMB, London (GB); Peng Tee KHAW, London (GB); Karen EASTLAKE, London (GB); Celia MURRAY-DUNNING, London (GB); Carla Patricia PINTO DE CARVALHO, London (GB)

(21) Appl. No.: 17/773,103

(22) PCT Filed: Oct. 30, 2020
(86) PCT No.: PCT/GB2020/052756

§ 371 Date: Apr. 29, 2022

(30) Foreign Application Priority Data
Oct. 31, 2019 (GB) 1915803.9

(51) Int. Cl. A61K 35/30; C12N 5/079; A61P 27/02

(52) U.S. Cl. CPC A61K 35/30; C12N 5/0622; A61P 27/02; C12N 2506/02; C12N 2500/98; C12N 2501/415; C12N 2501/41; C12N 2502/115; C12N 2501/11; C12N 2501/999; C12N 2501/998; C12N 2500/24

(57) ABSTRACT

The present invention relates to a novel process of producing therapeutic GMP grade Müller cells and Miller cells obtainable therefrom, derived from stem cells using products that are free of animal-derived components. The Müller cells are suitable for treatment of eye disease, including glaucoma. There is also provided a cell culture medium.

### FIELD OF THE INVENTION

The present invention relates to a novel process of producing therapeutic GMP grade Müller cells and Müller cells obtainable therefrom, derived from stem cells using products that are free of animal-derived components. The Müller cells are suitable for treatment of eye disease, including glaucoma. There is also provided a cell culture medium.

### BACKGROUND TO THE INVENTION

Glaucoma has become the most frequent cause of irreversible blindness worldwide and is estimated to affect ˜111 million people by 2040. Retinal ganglion cell (RGC) loss is the hallmark of optic neuropathies, including glaucoma, where damage to RGC axons occurs at the level of the optic nerve head (ONH). Under normal conditions RGCs receive visual signals from photoreceptors via the two preceding layers of neuronal cells (bipolar and amacrine cells) and transmit the information through axons which exit the eyeball via the ONH and optic nerve to the brain. Damage to RGCs occurs as a result of physical and molecular mechanisms including mechanical compression, decreased paracrine neurotrophic factor support, glial activation, oxidative stress/reduction in the antioxidant defence system, immune system dysregulation, and mitochondrial dysfunction/metabolic defects. These established mechanisms for RGC dysfunction, alongside emerging evidence in the literature, indicate an underlying, multifactorial, metabolic defect that results in the loss of RGC function and leads to RGC degeneration and death in glaucoma. Currently, approved treatments for glaucoma aim to slow down progression of the disease but ultimately these treatments do not prevent ongoing damage to RGCs and as such, the disease still progresses with many patients becoming blind in one or both eyes.

Several studies have highlighted the importance and key role of the Müller cell in providing functional and metabolic support to RGCs. Under homeostatic conditions Müller cells provide a multitude of beneficial functions including: provision of trophic substances and protection against neurotoxic glutamate, ion and water homeostasis, buffering of mechanical stimuli, structural stabilization of the retina, modulation of the immune and inflammatory response, antioxidant production, glucose metabolism, contain significant numbers of, and large, mitochondria indicating significant metabolic activity/support as well as facilitating significant neuroprotective levels of adenosine triphosphate (ATP), all of which are perturbed in glaucoma.

Based upon the known role of Müller cells, it is anticipated that a Müller cell therapy would, promote RGC repair, survival and function, and therefore improve visual function for patients suffering with optic neuropathies.

To evaluate this hypothesis a number of research studies have been conducted in relevant glaucoma-like animals models using Müller cells generated from different sources including from a human adult cadaveric donor eye retina (Singhal et al, Stem Cells Translational Medicine, 2012); from a feline source (Becker et al, Stem Cells Translational Medicine, 2016); and from a human iPSC line (Eastlake et al, Stem Cells Translational Medicine, 2019). Each of these pharmacology studies has shown that a single intravitreal administration of Müller cells can significantly improve RGC function.

The generation of embryoid bodies and formation of retinal organoids was based initially on the protocol of Nakano. Nakano et al (Cell Stem Cell 10, 771-785, Jun. 14, 2012) found that fetal bovine serum (FBS) is an effective enhancer for retinal differentiation of stem cells, however fetal bovine serum and other animal-based products are not acceptable in a cell-based therapeutic to be administered to humans.

There is therefore a global challenge to develop a culture and differentiation method to produce Müller cells in accordance with Good Manufacturing Practice (GMP) for clinical applications without negatively affecting the scalability, yield, morphology or biological properties of the cells. The present invention provides improved methods for culturing stem-cell derived GMP-compliant Müller cells suitable for cell therapy, and Müller cells derived therefrom.

### FIGURES

FIG. 1— Müller Cell. Schematic diagram showing Müller cell with other retinal cell types. GCL=ganglion cell layer. INL=inner nuclear layer. ONL=outer nuclear layer.

FIG. 2— Stage 2 of Cell Culture Process—Neural retina differentiation. Schematic diagram showing the initial process for stem cell differentiation to produce mature retinal organoids. Figure shows embryoid body formation from stem cells, induction of neural differentiation and maturation.

FIG. 3— Stage 3 of Cell Culture Process—Dissociation of Müller cells from retinal organoids and Müller cell propagation. Schematic diagram showing cell dissociation and propagation process. Müller cells are isolated from retinal organoids. Gentle Cell Dissociation Reagent (GCDR) dissociation can be used to create a single cell suspension, followed by centrifugation and plating on fibronectin coated flasks. Then follows feeding and expansion with FGF (fibroblast growth factor) and EGF (epidermal growth factor).

FIG. 4. Müller cells differentiated from RC-9 cells do not express stem marker, Tra-1-60. Expression of stem cell marker Tra-1-60 was measured using flow cytometry on the surface of undifferentiated RC-9 cells (FIG. 4A) and compared to expression on the surface of Müller cells differentiated from RC-9 cells (FIG. 4B) (produced using the GMP-compliant protocol). The undifferentiated RC-9 cells are highly positive for Tra-1-60 (99.44% of the population are positive), indicative of the stem cell status of the cells. Following differentiation of RC-9 cells to Müller cells, the expression of Tra-1-60 is lost.

FIG. 5. Müller cells differentiated from RC-9 cells express markers associated with Müller cells. Müller cells that had been differentiated from RC-9 cells using the GMP-compliant protocol were characterised for expression of Müller markers using flow cytometry. Vimentin (FIG. 5A and FIG. 5B), CD29 (FIG. 5C and FIG. 5D), CD44 (FIG. 5E and FIG. 5F) and Nestin (FIG. 5G and FIG. 511) were highly expressed by the derived Müller cells, when compared to the negative isotype control.

FIG. 6. Müller cells produced using GMP compliant protocol of the invention secrete neuroprotective factors and anti-oxidants, known to support RGC function. Müller cells that had been differentiated from RC-9 cells using the GMP-compliant protocol were characterised for secretion of neuroprotective factors and anti-oxidants using ELISA. BDNF, PEDF and PRDX6 concentrations were measured by ELISA in the supernatant from Müller cells and compared to undifferentiated (undiff) RC-9 hESCs (starting material); BLQ=below level of quantification.

FIG. 7. Müller cells produced using GMP compliant protocol of the invention express and secrete more BDNF than published cells. Müller cells that had been differentiated from RC-9 cells using the GMP-compliant protocol were characterised for expression of BDNF gene using transcriptomics and BDNF secretion in cell supernatant using ELISA. TPM=transcripts per million, B4=Müller cells of Eastlake et al. 2019, Eng1=Müller cells of the invention

FIG. 8. Müller cells produced using GMP compliant protocol of the invention have higher PEDF gene expression than published cells. Müller cells that had been differentiated from RC-9 cells using the GMP-compliant protocol were characterised for expression of PEDF gene expression using transcriptomics and PEDF secretion in cell supernatant using ELISA. TPM=transcripts per million, B4=Müller cells of Eastlake et al. 2019, Eng1=Müller cells of the invention.

FIG. 9. Müller cells produced using GMP compliant protocol of the invention do not express pluripotency markers. Müller cells that had been differentiated from RC-9 cells using the GMP-compliant protocol were characterised for expression of pluripotency markers using transcriptomics. Differential gene expression by DESeq in Bioconductor; numbers are normalised transcripts per million (TPM) reads. <10 TPM is below the level of quantification (BLQ). Undiff. RC-9=Undifferentiated RC-9 (starting material).

FIG. 10. Müller cells produced using GMP compliant protocol of the invention have lower POU5F1 (OCT3) gene expression than published cells. Müller cells that had been differentiated from RC-9 cells using the GMP-compliant protocol were characterised for expression of markers of pluripotency using transcriptomics. TPM=transcripts per million, B4=Müller cells of Eastlake et al. 2019, Eng1=Müller cells of the invention.

FIG. 11. Müller cells produced using GMP compliant protocol of the invention improve the survival of RGCs in vitro following treatment with excess glutamate, demonstrated by increased neurite length. Rat primary RGCs were treated with 25 μM glutamate for 24 hr and then treated with serum-free basal media (BM) or Müller cell (MC) supernatant (SN) for 72 hr. A, RGCs were immunostained with anti-β-tubulin III antibody (TUJ, Alexa 488, showing the shape of the cells) a marker of RGCs and DAPI (showing the nuclei). Scale bar=20 μm. B, Histogram plot shows the mean length of the primary neurite per RGC; error bars ±SEM; ** p<0.01; stats analysis Mann-Whitney Test. Data generated with Müller cells that had been differentiated from RC-9 cells using the GMP-compliant protocol of the invention.

FIG. 12. Müller cells differentiated from RC-9 improve RGC survival in vivo. The Müller cells of the invention were efficacious in an NMDA rodent model of RGC depletion and improved RGC function (measured by the scotopic negative threshold response of the electroretinogram- nSTR) in NMDA model measured by ERG, consistent with previous published data. A, 1×10⁵ Müller cells were intravitreally injected 1-wk following treatment with NMDA. B, Full ERG profile with nSTR highlighted (box) at −3.5 light intensity, the inset figure is the nSTR region only. Top line in nSTR box=NMDA control, middle line in nSTR box=NMDA+Müller cells, bottom line in nSTR box=Control. C, lowest point nSTR expressed as % RGC function at −3.5 light intensity; p=0.04. Left column=Control, middle column=NMDA control, right column=NMDA+Müller cells. Control=PBS treated eye; NMDA control=NMDA treated eye. Data generated using Müller cells derived from RC-9 hESCs.

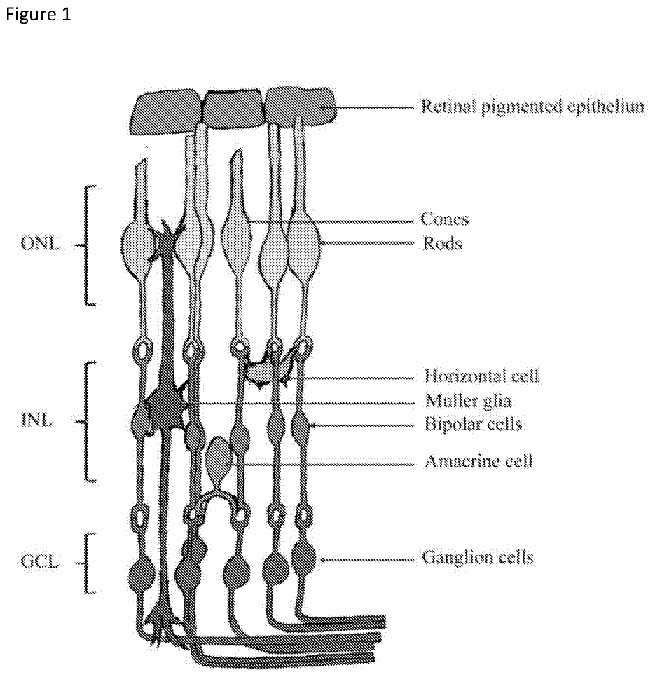

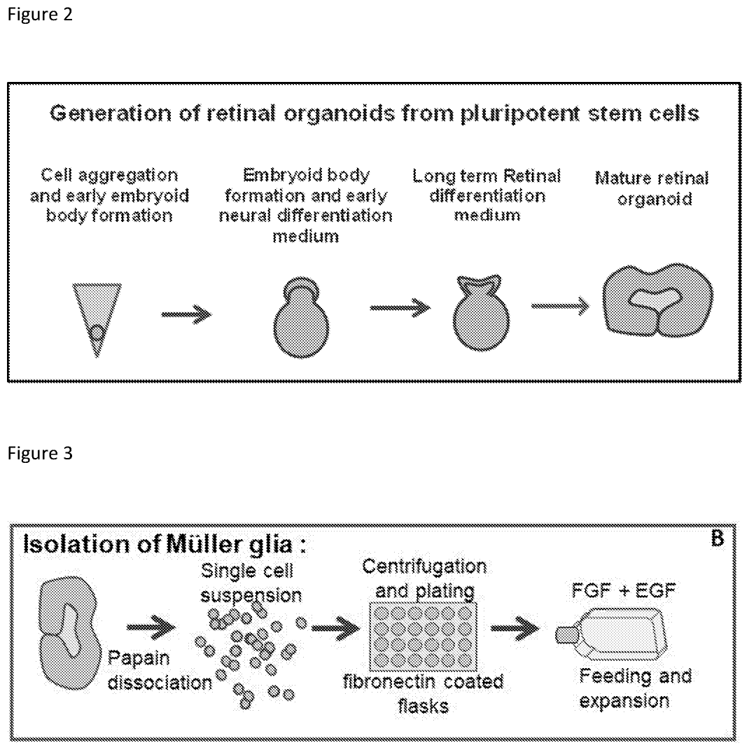

### SUMMARY OF THE INVENTION

The present invention is based on a novel process of producing Müller cells from stem cells using a culture process that is free of animal-derived components. Surprisingly in the process of conversion to a GMP-compliant method there has been no loss of yield or reduction in quality of the Müller cells. Cells cultured from pluripotent stem cells by the method of the present invention provide Müller cells that are morphologically like Müller cells derived from the iPSC BJ cell line described in Eastlake et al 2019 (Stem Cells Translational Medicine). Furthermore, the novel process of producing Müller cells from pluripotent stem cells using products that are free of animal-derived components is less labour-intensive, cost-effective, time-effective, lowers risk of infections, and is easier to scale up for industrial and clinical applications.

The invention also pertains to Müller cells derived from human embryonic stem cells (hESCs). In a preferred embodiment, the hESCs are RC-9 cells.

In the present disclosure, it is understood that in the context of Müller cells derived from the methods described herein, the terms “Müller” and “Müller-like” can be used interchangeably to describe cells that have been differentiated artificially to share the characteristics of Müller cells normally present in the eye.

Therefore, in an embodiment of the invention there is provided an isolated human Müller cell, wherein the cell:
a) expresses detectable levels of CD29, Vimentin, CD44 and Nestin and does not express detectable levels of Tra-1-60, and
b) is able to secrete the neurotrophins BDNF and PEDF.

In a further embodiment of the invention there is provided a purified, substantially homogenous, population of two or more Müller cells according to the invention.

In a further embodiment of the invention there is provided a population of human Müller cells, wherein at least 95% percent of the cells in the population express CD29, Vimentin, CD44 and Nestin to a detectable level and less than 5% of cells express Tra-1-60 to a detectable level, and wherein the cells are able to secrete the neurotrophins BDNF and PEDF.

In a further embodiment of the invention there is provided a Müller cell derived from human embryonic stem cells, wherein the cell:
a) expresses detectable levels of CD29, Vimentin, CD44 and Nestin and does not express detectable levels of Tra-1-60, and
b) is able to secrete the neurotrophins BDNF and PEDF.

In a further embodiment of the invention there is provided a method for producing therapeutic grade human Müller cells said method comprising,
a) culturing RC-9 human embryonic stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells.

In a further embodiment of the invention there is provided a pharmaceutical composition comprising the Müller cell of the invention, or the population of Müller cells according to the invention, or a Müller cell derivable from a method of the invention, and a pharmaceutically acceptable carrier.

In a further embodiment of the invention there is provided a pharmaceutical composition comprising a population of Müller cells obtainable from a method comprising:
a) culturing stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells; and a pharmaceutically acceptable carrier.

In a further embodiment of the invention there is provided a method of treating a retinal disease or condition, comprising administering the pharmaceutical composition according to the invention, the Müller cell according to the invention, the population of Müller cells according to the invention, or a Müller cell derivable from a method of the invention, to a patient in need thereof.

In a further embodiment of the invention there is provided a method of treating a retinal disease or condition comprising administering a pharmaceutical composition to a patient in need thereof, wherein the pharmaceutical composition comprises a pharmaceutically acceptable carrier and a population of Müller cells, wherein the Müller cells are obtained from a method comprising:
a) culturing stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells.

Therefore, in a further embodiment of the invention there is provided a method for producing therapeutic grade Müller cells said method comprising,
a) culturing stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells.

In a further embodiment the process involves a preliminary pre-differentiation stage wherein stem cells are cultured to 50-99% confluency, frozen at −80 degrees centigrade in a cryopreservation medium and then moved to liquid nitrogen for long-term storage and defrosted prior to step (a) above. The invention therefore includes a method for producing therapeutic grade Müller cells said method comprising culturing stem cells on a surface in a xeno- and serum-free medium to 50-99% preferably 50-80% confluency, freezing said cells in a cryopreservation medium and defrosting said cells, before culturing in accordance with the process above.

In a preferred embodiment the stem cells are human embryonic stem (hES) cells or induced pluripotent stem (iPS) cells preferably human embryonic stem cells. The stem cells used in the present invention are WA09 (hESC) from WiCell, Shef 1.3 (hESC), from University College London, Man-15 an hES line from University of Manchester, BJ cell line (iPSC) line from University College London, preferably RC-9 hES cell line provided by Roslin Cell Therapies.

In the process, the stem cells are on a surface that is coated, the preferred coating being a glycoprotein, combinations of laminin-111 and laminin-521, Matrigel™ or more preferably GMP-compliant synthetic vitronectin or synthetic vitronectin-based substrate.

The basal media for the process will include minimal essential medium such as GMEM with Glutamine and a synthetic media such as KOSR. The medium will be supplemented at each stage. For example during the pre-differentiation stage (prior to steps a)-d)) of growth of the stem cells, the medium can be TeSR-E8™ (animal component-free culture medium available from STEMCELL Technologies Inc.) but is preferably iPS-Brew which is a xeno-free cell culture medium, or TeSR2. The iPS-Brew can be supplemented with TGF beta (transforming growth factor beta). During the differentiation stages (steps a)-d) above) the basal media may include a carbon source such as sodium pyruvate, essential and non-essential amino acids and one or more antibiotics such as gentamycin, penicillin and/or streptomycin.

The adhesion promoter may be a synthetic matrix protein to avoid animal products and is preferably GMP-compliant synthetic vitronectin or synthetic vitronectin-based substrate.

The enriched growth factor is preferably synthetic or a product such as GMP-grade Human Platelet Lysate (HPL). At the stage of adding the enriched growth factor it is preferable to also add a neuronal differentiation enhancer such as an agonist of a protein of the hedgehog signalling pathway such as Smoothened protein (SAG).

Mammalian cells require iron for cell growth, DNA replication, cellular respiration, and metabolism. Transferrin is the natural physiological method by which iron is transported into the cell. In the final differentiation stages when the medium is supplemented with retinoic acid it is therefore preferable to also add human transferrin, such as a B27 supplement or N2 supplement which additionally contains human insulin, selenite, putrescine and progesterone that are important for cell survival.

In the process the stem cells are differentiated in suspension in a plate-based system preferably in a V-bottomed well plate, ideally 96 well plate.

The cell culturing may vary in time at each stage but the preferred culture protocol is to seed at day 0, to feed on days 2, 5 and 9 with the ROCK signalling pathway inhibitor and a Wnt inhibitor present preferably from 0 to at least 15 days, with the cell adhesion promoter added ideally from day 2 and/or for at least 8 days, and to feed again at day 12 and day 15 with the enriched growth factor and SAG present and/or for at least 2,3 or 4 days and to feed from day 17 with retinoic acid present and/or for at least 2 days. The ideal length of time for the retinal organoids to be visible from the embryoid body is 15-90 days preferably 15-70 days or at least 15 to 40 days. From this time the cells are ideally fed twice weekly with the medium including retinoic acid.

Retinal organoids arising from the embryoid body can be carefully dissected using microblades such as microscalpels or diamond tipped cutters between 25-40 days, to avoid damage to the cells under a dissection microscope, and transferred to new low adhesion plates in readiness for dissociation to harvest an enriched Müller cell suspension. The retinal organoids are maintained on the low adhesion plates in the medium including retinoic acid. These retinal organoids are harvested ideally between 30 and 300 days.

Between approximately 15-90 days embryoid bodies containing retinal organoids may be dissociated without dissection (allowing significantly greater scalability), in papain or a cell dissociation reagent such as Gentle Cell Dissociation Reagent (GCDR) from STEMCELL Technologies Inc, to isolate the enriched Müller cell suspension. Without being bound by theory this appears possible because the cells appear to grow much more densely in Human Platelet Lysate.

Therefore, in a further embodiment of the invention there is provided a method for producing Müller cells said method comprising,
a) culturing retinal organoids in human platelet lysate,
b) dissociating said retinal organoids without dissection to isolate Müller cells.

Müller cells are the only cells of the neural retina that express a CD29, ligand which binds to fibronectin hence it is advantageous to create a pure population of Müller cells by pre-coating the surface of a culture plate or flask with fibronectin allowing isolation of a pure population of a Müller cells (meaning pure from contamination of non-MUller cells, by removal of unwanted cells floating in the medium), and optionally expansion, most preferably in a medium supplemented with fibroblast growth factor (FGF) and epidermal growth factor (EGF) (the last step in FIG. 3) before creation of a bank of frozen cells in xeno-free media in vials.

In a further embodiment of the invention there is provided a method for producing a pure population of Müller cells said method comprising,
a) culturing retinal organoids,
b) dissociating said retinal organoids to isolate an enriched Müller cell suspension
c) culturing said Müller cells on a fibronectin coated surface
d) isolating said Müller cells on fibronectin to form a pure population of the same
e) optionally expanding said pure population of cells in a medium supplemented with fibroblast growth factor (FGF), or epidermal growth factor (EGF) or FGF and EGF.

In a further aspect of the invention there is provided a method for producing therapeutic grade pure human Müller cells said method comprising,
a) culturing stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate an enriched Müller cell suspension
f) culturing said Müller cells on a fibronectin coated surface
g) optionally expanding said pure population of cells in a medium supplemented with fibroblast growth factor (FGF) or epidermal growth factor (EGF), or FGF and EGF
h) isolating said Müller cells on fibronectin to form a pure population of the same.

According to a further aspect of the invention, Müller cells obtainable according to any of the methods outlined above are provided.

In a further aspect of the invention there is the use of Müller cells obtainable according to any of the methods outlined above in the manufacture of a medicament for the treatment of a condition associated with cell loss or cell damage, in particular eye diseases including but not limited to age-related macular degeneration, proliferative diabetic retinopathy, proliferative vitreoretinopathy, retinal detachment, retinitis pigmentosa, glaucoma and optic nerve injury and degeneration.

According to a further aspect of the invention, there is provided a cell culture medium for the differentiation of Müller cells (up to Day 15), consisting essentially of a minimum essential synthetic basal medium, a carbon source, non-essential amino acids a ROCK inhibitor, human platelet lysate, a Wnt inhibitor and a GMP-compliant synthetic vitronectin, synthetic vitronectin-based glycoprotein or hydrogel scaffold. A preferred medium for use (from approximately Day 12-18), contains an agonist of Smoothened protein of the hedgehog signalling pathway.

According to an further aspect of the invention, there is provided a cell culture medium for the differentiation of Müller cells (from Day 15) consisting essentially of a minimum essential synthetic basal medium, N2 supplement, retinoic acid, and human platelet lysate.

According to a further aspect of the invention, there is provided a cell culture kit for the culture of Müller cells comprising xeno- and serum-free medium as set out above.

### DETAILED DESCRIPTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a cell” includes “cells”, reference to “a tissue” includes two or more such tissues, reference to “a subject” includes two or more such subjects, and the like.

All publications, patents and patent applications cited herein, whether supra or infra, are hereby incorporated by reference in their entirety.

Stem cells have the ability to differentiate into various cell types in response to appropriate signals. These properties provide stem cells with capabilities for tissue repair, replacement, and regeneration. Accordingly, human stem cells more particularly human embryonic stem cells (hESCs) are of particular interest in medical research. Embryonic stem cells have the ability to differentiate into more cell types than adult stem cells hence have great potential in therapy. Differentiation is triggered by various factors in vivo, some of which can be replicated in in vitro stem cell cultures. Induced Pluripotent Stem Cells (iPSCs) are a form of stem cell, often used in autologous treatments as they can be created from the tissue of the same patient that will receive the transplant thus avoiding immune rejection. iPSCs obtained in this way do not have the ethical considerations of stem cells derived from embryos.

The nature of stem cells necessitates the use of special stem cell culture media and reagents. The culture is in a medium which is ideally a xeno- and serum-free medium. Xeno (non-human) and serum-free means there are no undefined animal products at all within the culturing process, which is important for GMP compliance. For example, FBS (fetal bovine serum) can be replaced with GMP quality HPL (human platelet lysate). Furthermore, Matrigel™ can be replaced by a human qualified Matrigel™ alterative such as Synthemax. Media useful in the invention include minimal essential media such as Glasgow Minimal Essential Medium (GMEM) optionally supplemented with L-glutamine and/or a synthetic media such as Knockout Serum replacement medium (KOSR). The media may be further supplemented with non-essential amino acids, growth promoters, carbon source such as sodium pyruvate, antibiotics such as penicillin and/or streptomycin, biological antioxidant such as 2-mercaptoethanol, Wnt signal pathway inhibitors more particularly Wnt antagonist IWR-1-endo, SAG (Smoothened agonist)which acts to enhance neuronal differentiation in human stem cells.

For cell culture in general most cells require a surface or an artificial substrate (adherent or monolayer culture) whereas others can be grown free-floating in culture medium (suspension culture). For the present invention stem cells are preferably cultured on a coated surface, the surface preferably being coated using a protein-based material, ideally glycoprotein because it improves both cell attachment and performance. The glycoprotein of choice is synthetic vitronectin because it acts as a substrate to promote attachment of cells through its binding domain RGD sequence (arginine, glycine and aspartate). Human recombinant vitronectin is preferred for GMP compliance and reduction of batch to batch variability. Matrigel™ is another cell adhesion promoter, but is not the preferred coating material. The cell culture media can be TeSR-E8™ but is preferably iPS-Brew with TGF beta, TeSR2 or StemPro. TeSR-E8™ is a feeder-free, animal component-free culture medium for human embryonic stem cells and human induced pluripotent stem (iPS) cells, and is available from STEMCELL Technologies Inc. iPS-Brew is a xeno-free cell culture medium that is commercially available from Miltenyi Biotec. The iPS-Brew can be supplemented with TGF beta.

Stem cell colonies are split upon reaching a certain level of confluency. “Confluence” or “confluency” refers to the percentage of the surface of a culture vessel or well that is covered by adherent cells. The culturing and expansion of the stem cells is preferable such that there are enough cells to seed for generation of retinal organoids.

The ability to freeze and defrost is important for quality control in large-scale preparation of clinical-grade cells. Stem cells that are sourced from suppliers are typically provided in a frozen state. A cryopreservation medium is used as part of the freezing process. Typical cryopreservation media support cells and prevent ice crystals from forming. The process of freezing and defrosting may be applied to stem cells before and/or after Stage 1 Maintenance. At the end of the complete process, when vials of Müller cells are produced, these may also be frozen for storage and subsequent defrosting at a later time.

After defrosting, the stem cells can be cultured further. The culturing can include passaging with multiple different media such as that described in the Examples section that follows (Stage 2 Differentiation).

The stem cells form embryoid bodies; three-dimensional aggregates of pluripotent stem cells. The embryoid bodies form a ‘mantle’ which is indicative of retinal organoids. ‘Organoids’ are self-organized three-dimensional tissue cultures that are derived from stem cells. Such cultures can replicate much of the complexity of an organ or to express selected aspects of it. Culturing is in low adhesion plates so the organoids are in suspension.

Retinal organoids are cultured for at least 15 days, and can be up to 300 days but is preferably 15 to 90 days, but this time frame may be 15-80 days, 15-70 days, 15-60 days, 15-50 days, 15-40 days or 15-30 days. The culture medium for support of retinal organoid production is critical to the invention and is supplemented with specific therapeutic grade reagents appropriate for the stage of production as set out above. A key reagent for production up to approximately day 15 is a ROCK signalling pathway inhibitor such as p160ROCK inhibitor and may be GMP-grade ROCK inhibitor, in preference to non-GMP ROCK inhibitor or research grade ROCK inhibitor. A further reagent included up to day 15 is a Wnt inhibitor which may also be either GMP grade or research grade.

A key reagent for production from approximately Day 2 is a synthetic cell adhesion promoter such as human recombinant vitronectin which may be used in preference to Matrigel™.

A further key reagent from approximately Day 12 is a synthetic enriched growth factor which may be GMP quality human platelet lysate (HPL) and an agonist of Smoothened protein of the hedgehog signalling pathway is preferably included from approximately Day15-18.

Further reagents ideally added to the medium from Day 15 include human transferrin such as N2 supplement and retinoic acid.

The retinal organoids can be isolated from the embryoid bodies using microblades. It is also possible to use other cutting tools such as blades, scalpels, microscalpels and/or diamond tipped cutters. From days 15-90 retinal organoids may be cut off from embryoid bodies under sterile microscopic conditions using microblades to purify optic cup structures from the embryoid bodies. A variation on this method is to dissect all organoids on one day between day 50 and day 57. The organoids can then be transferred to low adhesion plates and kept in medium for long term culture, with medium replacement twice a week.

The retinal organoids can then be dissociated to release Müller cells between 25 and 300 days from the start of the differentiation protocol. Dissociation can be achieved using papain-based protocols. Papain is a cysteine protease enzyme. A commercially available papain kit (Worthington Biochemical) can be used to dissociate organoids. A variation on the protocol is to completely replace the papain method by use of Gentle Cell Dissociation Reagent. Gentle Cell Dissociation Reagent (GCDR) is an enzyme-free reagent suitable for the dissociation of human embryonic stem cells or human induced pluripotent stem cells into small cell aggregates for routine passaging or into a single-cell suspension. GCDR is available from STEMCELL Technologies Inc.

The process produces therapeutic grade Müller cells when GMP-compliant reagents, such as xeno-and serum-free media are used. The resulting Müller cells are suitable for therapy in conditions associated with cell loss or cell damage, and in particular eye disease, including but not limited to age-related macular degeneration, proliferative diabetic retinopathy, proliferative vitreoretinopathy, retinal detachment, retinitis pigmentosa, glaucoma and optic nerve injury and degeneration.

The process described provides a cell culture media which may also be part of a cell culture kit.

This improved cell culture-based process generates therapeutic grade Müller cells without loss of yield or reduction in cell quality.

Thus, according to an further aspect of the invention, there is provided a cell culture medium for the differentiation of Müller cells (from Day 15), or retinal organoids harbouring an enriched population of Müller cells (from Day 15) consisting essentially of a minimum essential synthetic basal medium, N2 supplement, retinoic acid, and human platelet lysate.

Thus, according to a further aspect of the invention, there is provided a cell culture kit for the derivation of Müller cells or retinal organoids harbouring a rich population of Müller cells, comprising xeno- and serum-free medium as set out above.

### Müller Cells of the Invention

As set out above, the present invention also provides Müller cells. Preferably, the Müller cells are human cells. In an embodiment of the invention an isolated or purified human Müller cell is provided. The terms “purified” or “isolated” means separated from constituents, cellular and otherwise, in which the cell, tissue, polynucleotide, peptide, polypeptide, protein, antibody or fragment(s) thereof, which are normally associated in nature. A cell is isolated or purified if it is substantially free of any other components, such as culture medium and other cells. A purified or isolated cell is separated from tissue in which it is normally associated with in nature. An isolated or purified cell is a cell that is separated from tissue or cells of dissimilar phenotype or genotype. The Müller cells derived from the methods of the invention have the Müller cell marker identity described below. The Müller cells derived from the methods of the invention can be screened as described below for Müller cell markers to confirm their identity.

The Müller cells of the invention may advantageously be used to treat a disease in a subject. The Müller cells of the invention can be autologous or allogenic to the subject to be treated. The Müller cells of the invention are produced from stem cells according to the methods described herein, preferably from human stem cells, preferably from hESCs, preferably from RC-9 cells. The Müller cells derived from the methods described herein can be characterised for the marker profile set out below.

The Müller cells of the invention can be identified as Müller cells using standard methods known in the art, including expression of lineage restricted markers, and analysis of structural and functional characteristics. The Müller cells of the invention will express detectable levels of cell surface markers known to be characteristic of Müller cells. The Müller cells of the invention will express detectable levels of cell surface markers known to be characteristic of Müller cells when compared with undifferentiated stem cells. The Müller cells of the invention express detectable levels of CD29, Vimentin, CD44 and Nestin.

The Müller cells of the invention will not express detectable levels of cell surface markers known to be characteristic of undifferentiated stem cells. The Müller cells of the invention do not express detectable levels of Tra-1-60. In a preferred embodiment the Müller cells of the invention do not express detectable levels of Tra-1-60 and additionally do not express detectable levels of one or more of LIN28, SOX2, OCT3/OCT4, NANOG and ESRG. In a preferred embodiment the Müller cells of the invention do not express detectable levels of Tra-1-60 and additionally do not express detectable levels of one or more of LIN28, SOX2, OCT3/OCT4, NANOG, ESRG and DPPA4.

CD29, otherwise known as Integrin beta 1, VLA-β chain, or gpIIa. It acts as a fibronectin receptor and is involved in a variety of cell-matrix interactions. Integrin family members are membrane receptors involved in cell adhesion and recognition in a variety of processes including embryogenesis, haemostasis, tissue repair, immune responses and metastatic diffusion of tumor cells.

Vimentin is a type III intermediate filament (IF) protein that acts as a structural protein. It is expressed by many cells including mesenchymal cells and glial cells. Vimentin is responsible for maintaining cell shape, integrity of the cytoplasm, and stabilizing cytoskeletal interactions.

CD44 is a cell-surface glycoprotein involved in cell—cell interactions, cell adhesion and migration.

Nestin (neuroectodermal stem cell marker) is a type VI intermediate filament (IF) protein. Nestin is expressed in dividing cells during the early stages of neural development. It is downregulated upon neural maturation, and is also expressed in many other tissues. It is however mostly expressed in nerve cells where they are implicated in the radial growth of the axon. Nestin associates with Vimentin.

Tra-1-60 is a cell surface antigen expressed in undifferentiated human embryonic stem cells.

LIN28 is an RNA-binding protein that is highly expressed in human embryonic stem cells.

SOX2 is a transcription factor that is essential for maintaining pluripotency of undifferentiated embryonic stem cells.

OCT3/OCT4 (known either as OCT3, OCT4 or POU5F1) is a homeodomain transcription factor of the POU family. It is critically involved in the self-renewal of undifferentiated embryonic stem cells and is a marker of pluripotency.

NANOG is a transcription factor of the homeobox family that helps to maintain pluripotency of embryonic stem cells.

ESRG (embryonic stem cell related gene, also known as HESRG) is expressed specifically in undifferentiated human ESCs.

DPPA4 (Developmental Pluripotency Associated 4) is a highly specific marker of pluripotent cells.

The Müller cells of the invention are distinguished from known cells, including human embryonic stem cells, via their marker expression pattern. The Müller cells of the invention express detectable levels of CD29, Vimentin, CD44 and Nestin. The Müller cells of the invention preferably express an increased amount of these markers compared with human embryonic stem cells. The Müller cells of the invention preferably express an increased amount of all of the markers compared with human embryonic stem cells. This can be determined by comparing the expression level/amount of the markers in an Müller cell of the invention with the expression level/amount in a human embryonic stem cell using the same technique under the same conditions. Suitable hESCs are commercially available.

Müller cells of the invention do not express a detectable level of Tra-1-60. In a preferred embodiment the Müller cells of the invention do not express detectable levels of Tra-1-60 and additionally does not express detectable levels of one or more of LIN28, SOX2, OCT3/OCT4, NANOG and ESRG. In a preferred embodiment the Müller cells of the invention do not express detectable levels of Tra-1-60 and additionally does not express detectable levels of one or more of LIN28, SOX2, OCT3/OCT4, NANOG, ESRG and DPPA4.

Standard methods known in the art may be used to determine the detectable expression or increased expression of various markers discussed above (and below). Suitable methods include, but are not limited to, immunocytochemistry, immunoassays, flow cytometry, such as fluorescence activated cells sorting (FACS), polymerase chain reaction (PCR), such as reverse transcription PCR (RT-PCR), and transcriptomics methods such as RNA sequencing (RNA-Seq). Suitable immunoassays include, but are not limited to, Western blotting, enzyme-linked immunoassays (ELISA), enzyme-linked immunosorbent spot assays (ELISPOT assays), enzyme multiplied immunoassay techniques, radioallergosorbent (RAST) tests, radioimmunoassays, radiobinding assays and immunofluorescence. Western blotting, ELISAs and RT-PCR are all quantitative and so can be used to measure the level of expression of the various markers if present. The use of transcriptomics is disclosed in the Examples, wherein gene expression levels are set out as normalised transcripts per million (TPM) reads. Less than 10 TPM is defined as being below the level of quantification. The expression or increased expression of any of the markers disclosed herein is preferably done using flow cytometry. Antibodies and fluorescently-labelled antibodies for all of the various markers discussed herein are commercially-available.

The Müller cells of the invention have the functional characteristics of Müller cells and can express the genes and secrete the neurotrophin proteins BDNF and PEDF. The Müller cells of the invention can secrete the neurotrophins BDNF and PEDF and the antioxidant PRDX6. The ability of the Müller cells to secrete neurotrophins and anti-oxidants may be measured using standard assays known in the art. Suitable methods include, but are not limited to, enzyme-linked immunosorbent assays (ELISAs), flow cytometry and immunostaining. The Müller cells of the invention preferably secrete detectable levels of the neurotrophins BDNF and PEDF. The Müller cells of the invention preferably secrete an increased amount of both the neurotrophins BDNF and PEDF compared with an hESC.

The Müller cells of the invention are preferably capable of migrating to the retina and intercalating with cells such as rod and cone photoreceptors, bipolar cells and ganglion cells.

In a preferred embodiment of the invention a method for producing therapeutic grade human Müller cells is provided, said method comprising,
a) culturing human embryonic stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells,

wherein the Müller cells express detectable levels of CD29, Vimentin, CD44 and Nestin and do not express detectable levels of Tra-1-60, and are able to secrete the neurotrophins BDNF and PEDF.

Preferably, the human embryonic stem cells are RC-9 cells.

### Population of the Invention

The invention also provides a population of two or more Müller cells of the invention. Any number of cells may be present in the population. The population of the invention preferably comprises at least about 5×10⁵ Müller cells of the invention. The population more preferably comprises at least about 1×10⁶, at least about 2×10⁶, at least about 2.5×10⁶, at least about 5×10⁶, at least about 1×10⁷, at least about 2×10⁷, at least about 5×10⁷, at least about 1×10⁸ or at least about 2×10⁸ Müller cells of the invention. In some instances, the population may comprise at least about 1.0×10⁷, at least about 1.0×10⁸, at least about 1.0×10⁹, at least about 1.0×10¹⁰, at least about 1.0×10¹¹ or at about least 1.0×10¹² Müller cells of the invention or even more.

The population comprising two or more Müller cells of the invention may comprise other cells in addition to the Müller cells of the invention. However, at least 70% of the cells in the population are preferably Müller cells of the invention. More preferably, at least about 75%, at least about 80%, at least about 85%, at least about 90%, at least about 97%, at least about 98% or at least about 99% of the cells in the population are Müller cells of the invention.

The invention also provides specific populations of Müller cells.

The invention also provides substantially homogenous, purified population of Müller cells. “Substantially homogeneous” cell population describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype is determined by the markers of Müller cell identity described in more detail herein.

The invention provides a population of Müller cells, wherein at least 90%, 91%, 92%, 93%, 94%, 95%, 96%, 97%, 98% or preferably at least 99% percent of the cells in the population express CD29 to a detectable level and less than 10%, 9%, 8%, 7%, 6%, 5%, 4%, 3%, 2% or preferably less than 1% of cells express Tra-1-60 to a detectable level.

The invention provides a population of Müller cells, wherein at least 90%, 91%, 92%, 93%, 94%, 95%, 96%, 97%, 98% or preferably at least 99% percent of the cells in the population express Vimentin to a detectable level and less than 10%, 9%, 8%, 7%, 6%, 5%, 4%, 3%, 2% or preferably less than 1% of cells express Tra-1-60 to a detectable level.

The invention provides a population of Müller cells, wherein at least 90%, 91%, 92%, 93%, 94%, 95%, 96%, 97%, 98% or preferably at least 99% percent of the cells in the population express CD44 to a detectable level and less than 10%, 9%, 8%, 7%, 6%, 5%, 4%, 3%, 2% or preferably less than 1% of cells express Tra-1-60 to a detectable level.

The invention provides a population of Müller cells, wherein at least 90%, 91%, 92%, 93%, 94%, 95%, 96%, 97%, 98% or preferably at least 99% percent of the cells in the population express Nestin to a detectable level and less than 10%, 9%, 8%, 7%, 6%, 5%, 4%, 3%, 2% or preferably less than 1% of cells express Tra-1-60 to a detectable level.

The invention provides a population of Müller cells, wherein at least 90%, 91%, 92%, 93%, 94%, 95%, 96%, 97%, 98% or preferably at least 99% percent of the cells in the population express at least two of CD29, Vimentin, CD44 and Nestin to a detectable level and less than 10%, 9%, 8%, 7%, 6%, 5%, 4%, 3%, 2% or preferably less than 1% of cells express Tra-1-60 to a detectable level.

The invention provides a population of Müller cells, wherein at least 90%, 91%, 92%, 93%, 94%, 95%, 96%, 97%, 98% or preferably at least 99% percent of the cells in the population express at least three of CD29, Vimentin, CD44 and Nestin to a detectable level and less than 10%, 9%, 8%, 7%, 6%, 5%, 4%, 3%, 2% or preferably less than 1% of cells express Tra-1-60 to a detectable level.

In a preferred embodiment, the invention provides a population of Müller cells, wherein at least 90%, 91%, 92%, 93%, 94%, 95%, 96%, 97%, 98% or preferably at least 99% percent of the cells in the population express CD29, Vimentin, CD44 and Nestin to a detectable level and less than 10%, 9%, 8%, 7%, 6%, 5%, 4%, 3%, 2% or preferably less than 1% of cells express Tra-1-60 to a detectable level.

In a preferred embodiment, the invention provides a population of Müller cells, wherein at least 90%, 91%, 92%, 93%, 94%, 95%, 96%, 97%, 98% or preferably at least 99% percent of the cells in the population express CD29, Vimentin, CD44 and Nestin to a detectable level and less than 10%, 9%, 8%, 7%, 6%, 5%, 4%, 3%, 2% or preferably less than 1% of cells express Tra-1-60 to a detectable level, and additionally less than 10%, 9%, 8%, 7%, 6%, 5%, 4%, 3%, 2% or preferably less than 1% of cells express one or more of LIN28, SOX2, OCT3/OCT4, NANOG and ESRG to a detectable level.

In a preferred embodiment, the invention provides a population of Müller cells, wherein at least 90%, 91%, 92%, 93%, 94%, 95%, 96%, 97%, 98% or preferably at least 99% percent of the cells in the population express CD29, Vimentin, CD44 and Nestin to a detectable level and less than 10%, 9%, 8%, 7%, 6%, 5%, 4%, 3%, 2% or preferably less than 1% of cells express Tra-1-60 to a detectable level, and additionally less than 10%, 9%, 8%, 7%, 6%, 5%, 4%, 3%, 2% or preferably less than 1% of cells express one or more of LIN28, SOX2, OCT3/OCT4, NANOG, ESRG and DPPA4 to a detectable level.

The cells in these preferred populations may further express detectable levels of other markers with reference to Müller cells. The cells in these preferred populations may have any of the advantageous properties of the Müller cells discussed above.

In any of the embodiments above where populations are defined with reference to % of cells expressing certain markers, the populations preferably comprise at least 5,000 cells, such as at least 6,000, at least 7,000, at least 8,000, at least 9,000, at least 10,000, at least 20,000, at least 30,000, at least 40,000 cells, at least 50,000 cells, at least 100,000 cells, at least 200,000 cells, at least 250,000 cells or at least 500,000 cells. The populations more preferably comprise at least 5000 cells, at least 50,000 cells or at least 250,000 cells. These populations may comprise any of the number of cells discussed above.

The cells and populations of the invention are advantageous for therapy as discussed below. The cell or population of the invention may be isolated, substantially isolated, purified or substantially purified. A cell or population is isolated or purified if it is completely free of any other components, such as culture medium and other cells. A cell or population is substantially isolated if it is mixed with carriers or diluents, such as culture medium, which will not interfere with its intended use. Other carriers and diluents are discussed in more detail below. A substantially isolated or substantially purified cell or population does not comprise cells other than the Müller cells of the invention.

The Müller cells may be administered to the subject on one occasion. Alternatively, the Müller cells may be administered to the subject on at least two occasions, such as at least 3, at least 4, at least 5, at least 6, at least 7, at least 8, at least 9 or at least 10 occasions. The interval between the occasions can be defined by a suitably qualified professional.

### Pharmaceutical Compositions and Administration

The invention additionally provides a pharmaceutical composition comprising a Müller cell of the invention or a population of the invention in combination with a pharmaceutically acceptable carrier or diluent.

The term “pharmaceutically acceptable carrier” (or medium or diluent), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials maybe designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The various compositions of the invention may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19^{th} Edition, Mack Publishing Company, Eastern Pennsylvania, USA.

Compositions may be prepared together with a physiologically acceptable carrier or diluent. Typically, such compositions are prepared as liquid suspensions of cells. The cells may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions of the invention may contain minor amounts of auxiliary substances such as wetting or emulsifying agents, pH buffering agents, and/or adjuvants which enhance effectiveness.

In a preferred embodiment, a pharmaceutical composition is provided comprising a population of Müller cells obtainable from a method comprising:
(a) culturing human embryonic stem cells in suspension in a plate-based system in xeno- and serum-free medium in the presence of a ROCK signalling pathway inhibitor and a Wnt inhibitor for at least 15 days,
(b) supplementing the xeno- and serum-free medium of step (a) with a synthetic cell adhesion promoter and culturing said cells for at least 8 days,
(c) supplementing the xeno- and serum-free medium of step (b) with a synthetic enriched growth factor and an agonist of Smoothened protein of the hedgehog signalling pathway and culturing said cells for at least 3 days,
(d) culturing said cells from step (c) for an additional 2-300 days supplementing the xeno- and serum-free medium of step (c) with retinoic acid until retinal organoids are visible,
(e) dissociating said retinal organoids to isolate Müller cells; and a pharmaceutically acceptable carrier. In a preferred embodiment the human embryonic stem cells are RC-9 cells.

In a preferred embodiment the Müller cells of the pharmaceutical composition express detectable levels of CD29, Vimentin, CD44 and Nestin and do not express detectable levels of Tra-1-60, and are able to secrete the neurotrophins BDNF and PEDF. In a preferred embodiment the Müller cells of the pharmaceutical composition express detectable levels of CD29, Vimentin, CD44 and Nestin and do not express detectable levels of Tra-1-60, and additionally do not express detectable levels of one or more of LIN28, SOX2, OCT3/OCT4, NANOG and ESRG and are able to secrete the neurotrophins BDNF and PEDF. In a preferred embodiment the Müller cells of the pharmaceutical composition express detectable levels of CD29, Vimentin, CD44 and Nestin and do not express detectable levels of Tra-1-60, and additionally do not express detectable levels of one or more of LIN28, SOX2, OCT3/OCT4, NANOG, ESRG and DPPA4 and are able to secrete the neurotrophins BDNF and PEDF.

The Müller or Müller cells of the invention, the population of the invention, or the pharmaceutical composition of the invention are administered in a manner compatible with the dosage formulation and in such amount will be therapeutically effective. The quantity to be administered depends on the subject to be treated. Precise amounts of Müller cells required to be administered may depend on the judgment of the practitioner and may be peculiar to each subject.

Any suitable number of cells may be administered to a subject. For example, at least, or about, 0.2×10⁶, 0.25×10⁶, 0.5×10⁶, 1.5×10⁶, 4.0×10⁶ or 5.0×10⁶ cells per kg of subject may administered. For example, at least, or about, 10⁵, 10⁶, 10⁷, 10⁸, 10⁹ cells may be administered. As a guide, the number of cells of the invention to be administered may be from 10⁵ to 10⁹, preferably from 10⁶ to 10⁸. Typically, up to 2×10⁸ Müller cells are administered to each subject. Any of the specific numbers discussed above with reference to the populations of the invention may be administered. In such cases where cells are administered or present, culture medium may be present to facilitate the survival of the cells. In some cases the cells of the invention may be provided in frozen aliquots and substances such as DMSO may be present to facilitate survival during freezing. Such frozen cells will typically be thawed and then placed in a buffer or medium either for maintenance or for administration.

The cells may be administered via any reasonable route, such as intraocularly, intravitreally or subretinally.

### Medicaments, Methods and Therapeutic Use

The Müller cells of the invention, the populations of the invention, or the pharmaceutical compositions of the invention may be used in a method of therapy of the human body. Thus the invention provides a Müller cell of the invention, a population of the invention, or a pharmaceutical composition of the invention for use in a method of treatment of the human body by therapy. In particular, the invention concerns using the Müller cells of the invention, a population of the invention, or the pharmaceutical composition of the invention to treat disease, such as a retinal disease or condition.

In a preferred embodiment a method of treating a retinal disease or condition is provided comprising administering a pharmaceutical composition to a patient in need thereof, wherein the pharmaceutical composition comprises a pharmaceutically acceptable carrier and a population of Müller cells, wherein the Müller cells are obtained from a method comprising:
(a) culturing human embryonic stem cells in suspension in a plate-based system in xeno- and serum-free medium in the presence of a ROCK signalling pathway inhibitor and a Wnt inhibitor for at least 15 days,
(b) supplementing the xeno- and serum-free medium of step (a) with a synthetic cell adhesion promoter and culturing said cells for at least 8 days,
(c) supplementing the xeno- and serum-free medium of step (b) with a synthetic enriched growth factor and an agonist of Smoothened protein of the hedgehog signalling pathway and culturing said cells for at least 3 days,
(d) culturing said cells from step (c) for an additional 2-300 days supplementing the xeno- and serum-free medium of step (c) with retinoic acid until retinal organoids are visible,
(e) dissociating said retinal organoids to isolate Müller cells. In a preferred embodiment the human embryonic stem cells are RC-9 cells.

In a preferred embodiment the Müller cells of the pharmaceutical composition express detectable levels of CD29, Vimentin, CD44 and Nestin and do not express detectable levels of Tra-1-60, and are able to secrete the neurotrophins BDNF and PEDF. In a preferred embodiment the Müller cells of the pharmaceutical composition express detectable levels of CD29, Vimentin, CD44 and Nestin and do not express detectable levels of Tra-1-60, and additionally do not express detectable levels of one or more of LIN28, SOX2, OCT3/OCT4, NANOG and ESRG and are able to secrete the neurotrophins BDNF and PEDF. In a preferred embodiment the Müller cells of the pharmaceutical composition express detectable levels of CD29, Vimentin, CD44 and Nestin and do not express detectable levels of Tra-1-60, and additionally do not express detectable levels of one or more of LIN28, SOX2, OCT3/OCT4, NANOG, ESRG and DPPA4 and are able to secrete the neurotrophins BDNF and PEDF.

In some embodiments, the retinal disease or condition is vision loss, blindness, glaucoma, optic nerve damage, optic nerve degeneration, a disease that cause optic nerve damage or degeneration, dominant optic atrophy, Leber hereditary optic neuropathy, congenital amaurosis, optic neuritis, a mitochondrial disorder that causes optic nerve damage, a ganglion cell disease, an optic nerve cell disease, or ischemic optic neuropathy.

In some embodiments the glaucoma is:
(a) primary glaucoma, including primary open angle glaucoma, acute primary angle closure glaucoma, chronic primary angle closure glaucoma, normal tension glaucoma, childhood glaucoma and juvenile glaucoma; or
(b) secondary glaucoma, including developmental glaucoma such as Axenfeld anomaly, Rieger anomaly, Reiger syndrome, Aniridia and Peters anomaly, traumatic glaucoma, steroid induced glaucoma, pseudoexfoliative glaucoma, pigmentary glaucoma, uveitic glaucoma, neovascular glaucoma, mixed mechanism glaucoma, irido corneal endothelial syndrome, a disease causing optic nerve injury, Posner-Schlossman syndrome, juvenile rheumatoid arthritis and Ankylosing spondylitis with secondary uveitis.

In some embodiments the Müller cell of the invention, a population of the invention, or a pharmaceutical composition of the invention are used in the treatment of a condition associated with cell loss or cell damage. The condition associated with cell loss or cell damage may be, for example, age-related macular degeneration, proliferative diabetic retinopathy, proliferative vitreoretinopathy, retinal detachment, retinitis pigmentosa, glaucoma or optic nerve injury and degeneration.

The invention also provides a Müller cell of the invention, a population of the invention, or a pharmaceutical composition of the invention for use in the manufacture of a medicament for the treatment of the diseases or conditions described herein.

The invention also provides a Müller cell of the invention, a population of the invention, or a pharmaceutical composition of the invention for use in the treatment of the diseases or conditions described herein.

### Examples

### Example 1

Stage 1: Maintenance of RC-9 hES (Human Embryonic Stem) Cells

Maintenance: PSC (pluripotent stem cell) cultures were maintained as feeder free colonies on 6 well plates coated with human ESC qualified Matrigel™ in a volume of 2 mL of TeSR-E8™ media supplemented with 50 μM gentamycin or penicillin/streptomycin. Matrigel™ is the trade name for a gelatinous protein mixture secreted by Engelbreth-Holm-Swarm (EHS) mouse sarcoma cells produced and marketed by Corning Life Sciences and BD Biosciences. The main components of Matrigel™ are type IV collagen, laminin, heparan sulfate proteoglycan and entactin.

Cell lines were observed daily to check for PSC-like morphology, the presence of differentiated cells and confluence. Any differentiated cells were removed using a 200 μL pipette tip and aseptic conditions under an EVOS XL Core microscope in a tissue culture hood. Other microscopes can be used if they have a good ×40 objective and can be used within a culture hood.

Cells were fed daily by carefully aspirating media from the wells and by gently adding 2 mL of fresh TeSR-E8™ media containing gentamycin or penicillin/streptomycin. Weekend feeding consisted of a media change on Friday to Essential 8 Flex media, which was then replaced with TeSR-E8™ media on the Monday. An alternative to this method is using iPS Brew with TGF-b and carrying out a triple feed on Friday.

Propagation: Upon daily observation, colonies with well-defined edges were split at a ratio of 1:6 when approximately 70% confluent. To achieve this media was aspirated from each well, and cells were firstly washed with 1 mL of PBS (phosphate-buffered saline). Then 1 mL of 0.5 mM EDTA (ethylenediaminetetraacetic acid) in PBS was then added, and cells incubated for 4-6 minutes at room temperature until small holes appeared throughout the colonies. The EDTA solution was then aspirated and cells were gently washed from the plate by adding 2 mL of TeSR-E8™ media and repeated gently pipetting to dislodge colonies. The cell suspension was then diluted to the required split ratio and seeded onto Matrigel™ coated 6 well plates.

A variation on this method is to use vitronectin coated 6 well plates, instead of Matrigel™ coated plates. Another variation on this method is to use iPS-Brew and TGF beta, instead of TeSR-E8™ and Essential 8 Flex media. An additional variation is to use TrypLE to remove the cells, rather than EDTA.

Freezing PSC. Upon the cells reaching 70% confluency, medium was aspirated and cells were washed with 1 mL of PBS. Cells were then dissociated using 1 mL of 0.5 mM EDTA in PBS by incubation for 4-6 minutes at room temperature (as above). After aspirating the EDTA, cells were gently dissociated using mFreSR™ cryopreservation medium. mFreSR™ is a defined, serum-free cryopreservation medium designed for the cryopreservation of human embryonic and induced pluripotent stem cells (iPSCs). mFreSR™ includes dimethylsulfoxide (DMSO). Each 6 well plate produces enough cells for 6 vials of 1 mL cell suspension in mFreSR™ cryopreservation medium. The vials containing 1 mL of cell suspension were then frozen at −80° C. using a cell freezing container filled with isopropanol, before transfer to liquid nitrogen for long term storage.

A variation on this method is to replace mFreSR™ cryopreservation medium with Stemcell Technologies CS10 or CS2. CS10 and CS2 are a serum-free, animal component-free, and defined cryopreservation media containing 10% or 2% dimethyl sulfoxide (DMSO) respectively.

Defrosting PSC. Matrigel™ coated 6-well plates were prepared for 1 hour at room temperature prior to cell defrosting. A vial of cells was thawed for 3 minutes using a 37° C. water bath. The cell suspension was then collected with 2 mL of TeSR-E8™ media containing 10 μM ROCKi (Rock inhibitor) and centrifuged at 300 g to obtain a cell pellet. The supernatant was removed and the cell pellet resuspended in 2 mL of TeSR-E8 containing 10 μM ROCKi and 50 μM gentamicin for plating into the pre-coated Matrigel™ wells for culture.

A variation on this protocol is to use iPS-Brew without ROCKi instead of TeSR-E8. Another variation is to use vitronectin rather than Matrigel™. An additional variation is to use penicillin/streptomycin rather than gentamicin, or not to use any type of antibiotic.

Stage 2: Differentiation

A method based on that of Tokushige Nakano et al. (Cell Stem Cell 10, 771-785, Jun. 14, 2012) is used to generate retinal organoids. Nakano does not describe Müller glia identification but there have been reports in other settings; Xiufeng Zhong, et al. Nature Communications. Volume 5, Article number: 4047 (2014) and Chen HY et al. Molecular Vision. 9 Sep. 2016, 22:1077-1094.

Three different media are used in Stage 2 as follows.

Medium 1
384.5 mL GMEM (Glasgow minimum essential medium) with L-glutamine
100 mL KOSR (knockout serum replacement)
5 mL of 100×sodium pyruvate
5 mL of 100×non-essential amino acids
5 mL of 100×penicillin/streptomycin

Medium 2
334.5 mL GMEM with L-glutamine
100 mL of KOSR
50 mL of HPL
5 mL of 100×sodium pyruvate
5 mL of 100×non-essential amino acids
5 mL of 100×penicillin/streptomycin

Medium 3
439.5 mL DMEM(Dulbecco's Modified Eagle Medium)/F12-Glutamax
50 mL of HPL
5 mL of 100×N2 supplement
5 mL of 100×penicillin/streptomycin/amphotericin
0.5 μM retinoic acid

Other factors added to the media at various concentrations indicated in the text below include:
ROCK inhibitor in Medium 1, 20 μM
Wnt antagonist IWR-1-endo in Medium 1, 3 μM
SAG (smoothened agonist), in Medium 2, 100 nM
Human qualified Matrigel™, in Medium 1 at Day 2, 2% (w/v) Matrigel™ (final concentration of Matrigel™ 1%) and from Day 5 —18, 1% (w/v) Matrigel™.

In a variation of the above media the antibiotics can be omitted.

In a variation of the above media, the human qualified Matrigel™ alternative is Synthemax.

Day 0 Feeding

PSC which had been grown into confluent monolayers in 6-well plates, as indicated above, were used for differentiation of retinal organoids. Upon confluence, cells were washed 1× with PBS and dissociated with 1 mL TrypLE (×1) trypsin replacement containing 10 μM ROCKi and 0.5 mg/mL DNase at 37° C. Inactivation of TrypLE was performed by addition of 5 mL of ‘Medium 1’. Cells were then pelleted by centrifugation at 300 g for 5 minutes.

The supernatant was discarded and cells were suspended in 2 mL of ‘Medium 1’ containing 10 μM ROCKi for cell counting. Cells were then adjusted to a concentration of 9×10⁴/mL in ‘Medium 1’ containing 20 μM ROCKi and 3 μM Wnt antagonist. Aliquots of 100 μL (9,000 cells) were placed in each well of a 96-well V-bottomed plate and cells were cultured at 37° C., 5% CO2, atmospheric 02.

Day 2, 5, 9 Feeding

After 2 days in culture, 100 μL of ‘Medium 1’ containing 20 μM ROCKi, 3 μM Wnt antagonist and 2% (w/v) Matrigel™ (final concentration of Matrigel™ 1%) were added to each well of the 96-well V-bottomed plates.

Partial change of medium was performed on days 5 and 9, by removal of 100 μL medium and replacement with 100 μL of ‘Medium 1’ containing 20 μM ROCKi, 3 μM Wnt antagonist and 1% (w/v) Matrigel™.

Day 12 Feeding

The embryoid bodies (EBs) formed were gently transferred into each well of a 25-well squared, low adhesion plate using a 1,000 μL pipette tip, with the tip cut to avoid any damage to the EBs. Individual EBs were moved with approximately 100 —200 μL of medium. Each well was then topped up with 1 mL of ‘Medium 2’ containing 1% (w/v) Matrigel™ and 100 nM SAG. Plates were then cultured at 37° C. as described above.

Day 15 Feeding

Medium was replaced with fresh ‘Medium 2’ containing 1% (w/v) Matrigel™ and 100 nM SAG. The EBs were examined under an inverted microscope for the clear appearance of a ‘mantle’, which is indicative of retinal organoids and is visible from day 15.

Day 18 and Onwards Feeding

Medium was removed from each well and replaced with ‘Medium 3’. The EBs were fed twice weekly.

From days 15-90 retinal organoids were cut off under microscopic conditions using microblades. This procedure was carried out to purify optic cup structures from EBs. The organoids were then transferred to new 25-well squared low adhesion plates and kept in ‘Medium 3’ for long term culture, with medium replacement twice a week. Müller cells were isolated from retinal organoids between 30 and 300 days from the start of the differentiation protocol.

A variation on this method is to dissect all organoids on one day between day 50 and day 57.

Stage 3: Differentiation and Propagation

Retinal organoids containing human Müller cells were harvested between 30 and 300 days after initiation of retinal differentiation. A variation on this method is to dissociate all organoids on day 70.

Organoids were dissociated using a papain dissociation kit protocol supplied by the manufacturers (Worthington Biochemical) and used as per manufacturer's instructions. Single or multiple pooled organoids were isolated using this protocol.

Firstly 32 mL Earle's Balanced Salt Solution (EBSS) (vial 1) was added to the albumin ovomucoid inhibitor mixture (vial 4) and the contents allowed to dissolve while preparing the other components. Then 5 mL EBSS (vial 1) was added to a papain vial (vial 2) and placed in a 37° C. water bath for ten minutes or until the papain was completely dissolved. The solution was used promptly at room temperature during the dissociation.

A volume of 500 μL EB SS was added to the DNase vial (vial 3) and mixed gently. 250 μL of this solution was added to the vial containing the papain. This preparation contained a final concentration of approximately 20 units/mL papain and 0.005 DNase. Tissue was placed in the papain solution. The vial containing the tissue was placed at 37° C. with agitation by pipetting up and down every 10 mins for 30 min to 1.5 hours.

Following the papain incubation, the mixture was then triturated with a 1 mL pipette. Non-dissociated tissue remaining after trituration was allowed to settle to the bottom of the tube. The cloudy cell suspension was carefully removed and placed in sterile screw capped tube and centrifuged at 300 g for 5 minutes at room temperature. The supernatant was discarded and the cell pellet was immediately resuspended in the DNAse dilute albumin-inhibitor solution [2.7 mL EBBS (Earle's balanced salt solution) (vial 1); 300 μL of reconstituted albumin-ovomucoid inhibitor solution (vial 4); 150 μL of DNAse solution (vial 3)].

The discontinuous density gradient was then prepared as follows. A volume of 5 mL of albumin-inhibitor solution (vial 4) was added to a centrifuge tube, carefully layering the cell suspension on top and centrifuging at 70 g for 6 minutes at room temperature. The interface between the two layers of the gradient is clearly visible. The supernatant is discarded, leaving a cell pellet.

A variation on this method is to use Gentle Cell Dissociation Reagent, in place of the papain dissociation kit as supplied for example by Worthington Biochemical. In this method using Gentle Cell Dissociation Reagent, add 200 μL per organoid, triturate and place into an incubator. Triturate every 5 minutes for usually only 10 minutes, but this can be up to 2 hours for full dissociation.

Following dissociation from retinal organoids, cells were centrifuged to obtain a pellet, immediately resuspended in cell culture medium consisting of DMEM (Dulbecco's Modified Eagle Medium) containing 10% (v/v) FBS, 20 ng/mL FGF and 20 ng/mL EGF. Plates and flasks used to culture Müller cells were pre-coated with fibronectin (50 μg/mL for 2 hours at 37° C.).

Cells isolated from a single retinal organoid were initially cultured in a well of a 24-well plate and grown to confluence before passaging into a single well of a 6-well plate. Upon reaching confluence in the 6-well plate, cells were transferred onto a T25 tissue culture flask and subsequently into a T75 culture flask. Later passages were consistently performed in T75 flasks at 1:3 dilutions until enough cells for gene and protein expression analyses were obtained.

From the isolation date, it takes approximately 1-2 weeks to obtain approximately 1×10⁶ cells. The inventors also have evidence that the yield of Müller cell culture and expansion using the GMP-compliant method of the invention is significantly higher and the speed is faster (yield of ˜1e6 cells per colony in 7 days, as extrapolated by pooling, and ˜60e6 cells within 14 days) than the non-GMP/research-grade method (yields ˜1e6 cells after ˜28-30 days). Cell number could be increased by an order of 10 if necessary.

This was further expanded over another 4 weeks to generate a bank of frozen vials of 1×10⁶ cells per vial.

### Example 2—Cell Characterisation

The present inventors have derived allogeneic Müller cells from hESCs (RC-9 cell line) using the optimised GMP-compliant method of the invention and shown that these cells
express key Müller markers
do not express stem cell markers
secrete key neurotrophins BDNF and PEDF
increase the survival of RGCs in vitro
Increase the function of RGCs in vivo.

Müller cells differentiated from RC-9 cells do not express stem marker, Tra-1-60.

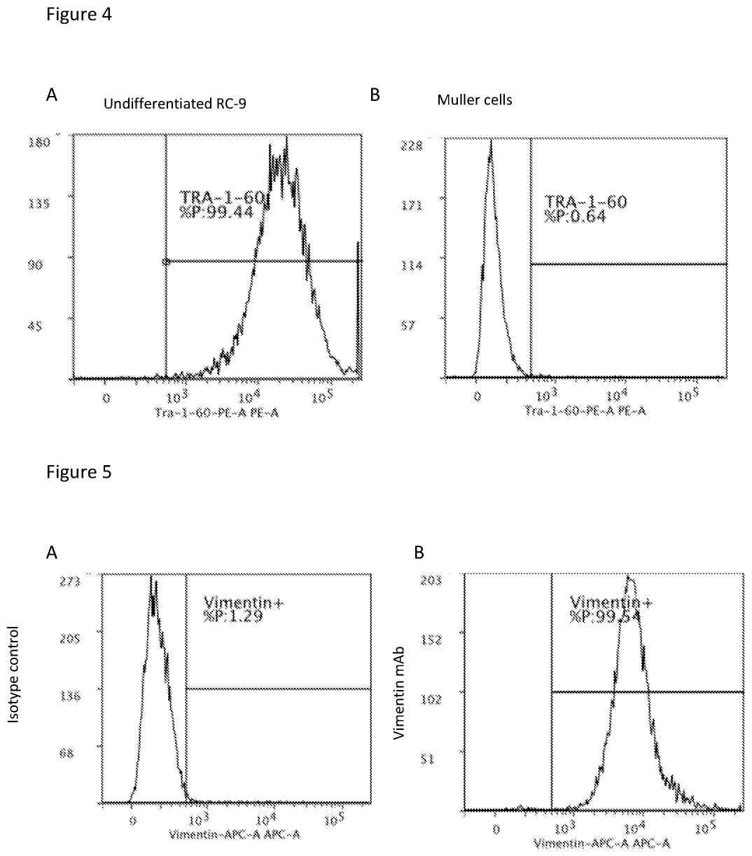

Expression of stem cell marker Tra-1-60 was measured using flow cytometry on the surface of undifferentiated RC-9 cells and compared to expression on the surface of Müller cells differentiated from RC-9 cells (produced using the GMP-compliant protocol). The undifferentiated RC-9 cells are highly positive for Tra-1-60 (99.44% of the population are positive), indicative of the stem cell status of the cells. Following differentiation of RC-9 cells to Müller cells, the expression of Tra-1-60 is lost as shown in FIG. 4.

Müller cells differentiated from RC-9 cells express markers associated with Müller cells.

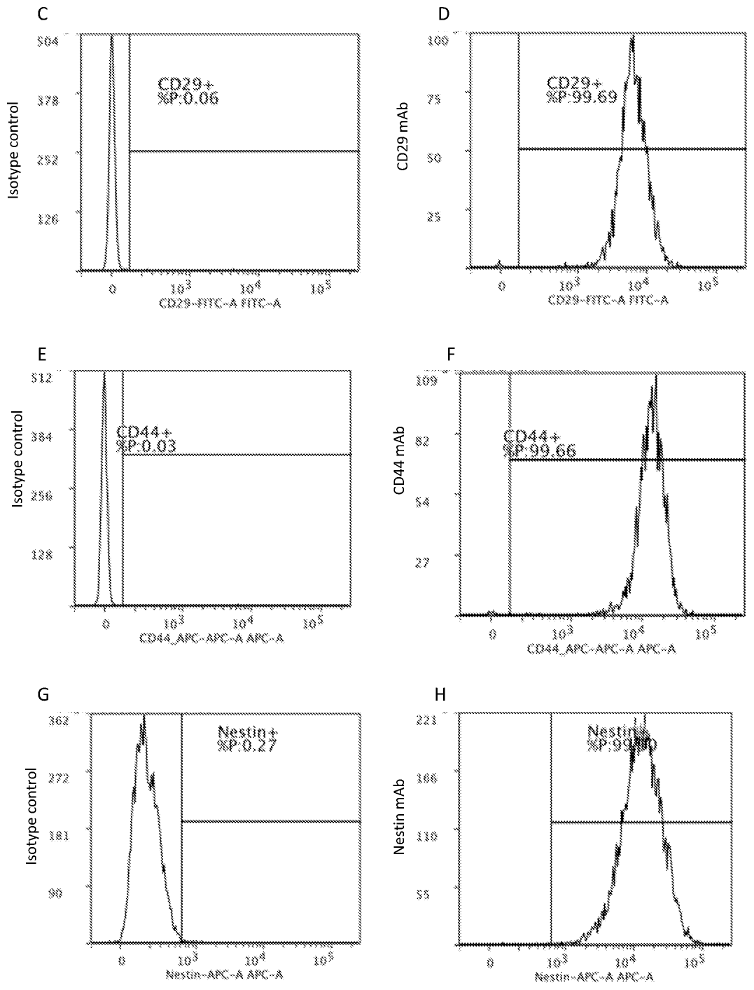

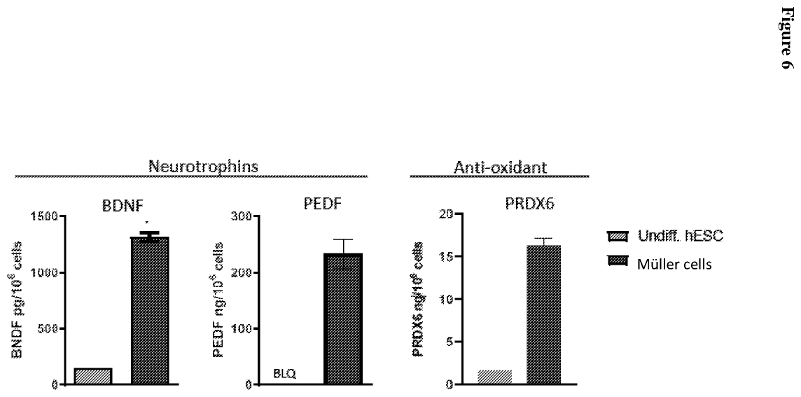

Müller cells that had been differentiated from RC-9 cells using the GMP-compliant protocol were characterised for expression of Müller markers using flow cytometry. Vimentin, CD29, CD44 and Nestin were highly expressed by the derived Müller cells, when compared to the negative isotype control. FIG. 5 shows that Müller cells differentiated from RC-9 cells express markers associated with Müller cells.

Müller cells differentiated from RC-9 cells secrete neuroprotective factors and anti-oxidants, known to support RGC function, to a greater extent than differentiated hESCs.

Müller cells that had been differentiated from RC-9 cells using the GMP-compliant protocol were characterised for secretion of neuroprotective factors and anti-oxidants using ELISA. BDNF, PEDF and PRDX6 concentrations measured by ELISA in the supernatant from Müller cells where significantly higher than the concentrations seen in undifferentiated RC-9 hESCs.

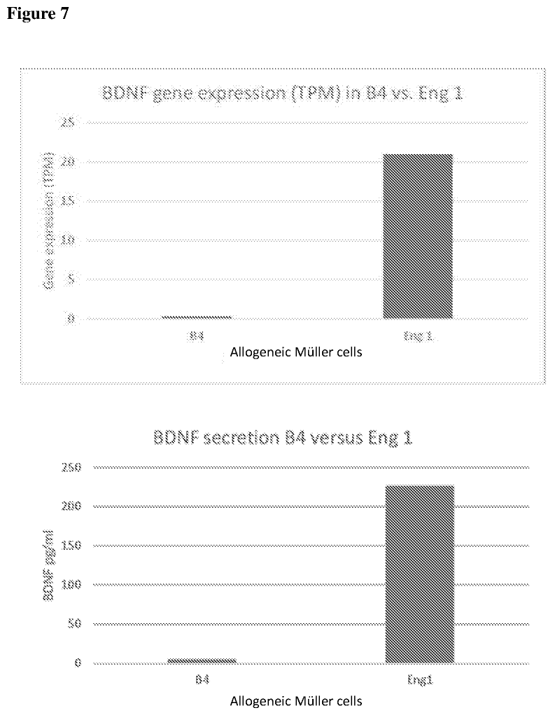

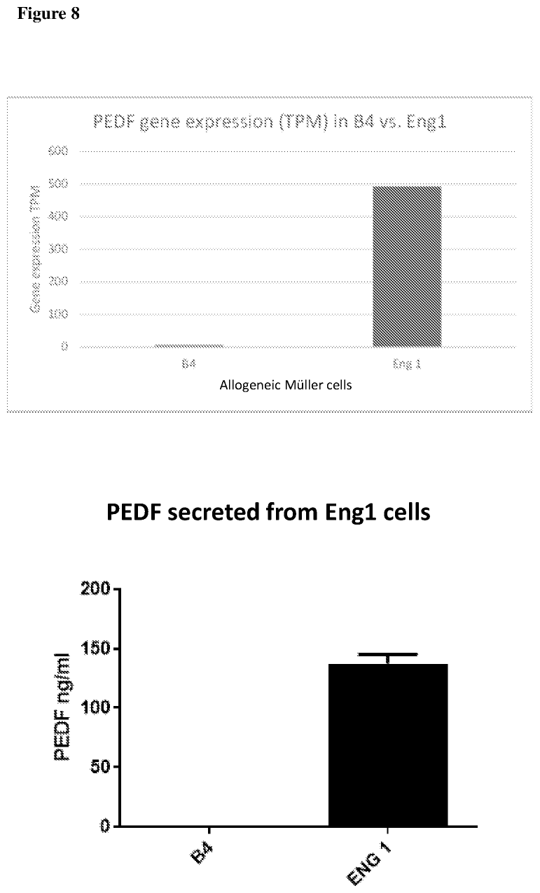

Müller cells differentiated from RC-9 cells express and secrete markers associated with Müller cells to a greater extent than Müller cells differentiated from iPS cells. Müller cells that had been differentiated from RC-9 cells using the GMP-compliant protocol were characterised for expression of Müller markers using transcriptomics and quantification of secretion levels in cell supernatant using ELISA. Müller cells produced using GMP compliant protocol of the present invention expressed and secreted more BDNF (FIG. 7), and expressed more PEDF (FIG. 8) than the published cells of Eastlake et al. 2019, which were differentiated from iPS cells.

Thus, the Müller cells of the present invention both express genes for BDNF and PEDF— two key neurotrophins that are related to the mechanism of action of Müller cells, but also express the key neurotrophins to a greater extent than the Müller cells known in the art that have been produced by other means.

Müller cells differentiated from RC-9 cells do not express pluripotency markers LIN28, SOX2, OCT3/4, NANOG, ESRG and DPPA4.

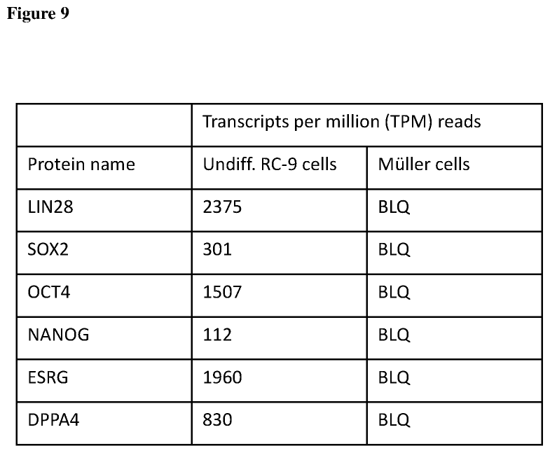

Expression of pluripotency markers, LIN28, SOX2, OCT3/4, NANOG, ESRG and DPPA4 was measured using transcriptomics in undifferentiated RC-9 cells and compared to expression on the surface of Müller cells differentiated from RC-9 cells (produced using the GMP-compliant protocol). The undifferentiated RC-9 cells were highly expressing all the markers, indicative of the stem cell status of the cells. Following differentiation of RC-9 cells to Müller cells, the expression of all the markers was lost, as shown in FIG. 9.

Müller cells differentiated from RC-9 express the marker, POU5F1 (OCT3) to a much lesser extent than Müller cells differentiated from iPS cells.

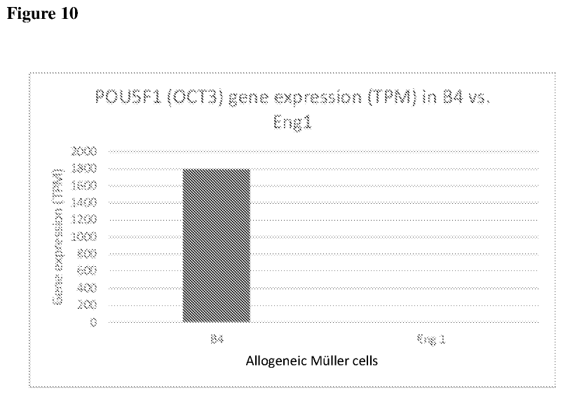

Gene expression of the pluripotency marker OCT3 was measured using transcriptomics in Müller cells differentiated from iPS cells following the protocol of Eastlake et al. 2019 and compared to expression in Müller cells differentiated from RC-9 cells (produced using the GMP-compliant protocol of the present invention). The Müller cells differentiated from iPS cells following the protocol of Eastlake et al. 2019 expressed a significantly higher level of OCT3 than the Müller cells of the invention (FIG. 10). OCT3 is a marker of pluripotency that is expected to be decreased following differentiation. Thus products with lower levels of this gene/protein would thus be considered to be safer to administer to patients.

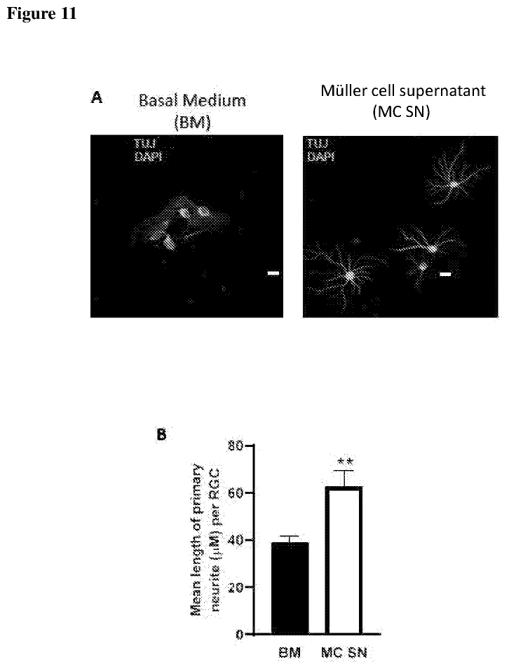

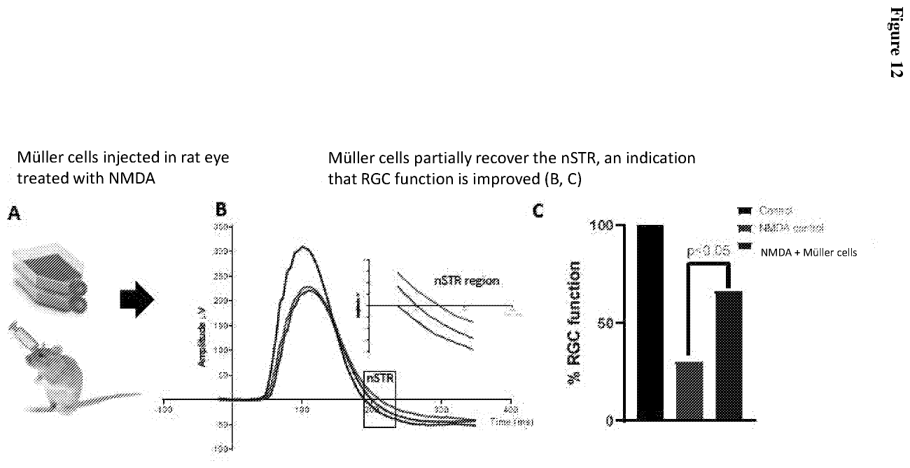

Müller cells differentiated from RC-9 improve RGC survival in vitro. Müller cells differentiated from RC-9 cells and produced using the GMP compliant protocol of the invention improve the survival of RGCs following treatment with excess glutamate, demonstrated by increased neurite length (FIG. 11A and B).

Müller cells differentiated from RC-9 improve RGC survival in vivo. Müller cells of the invention were efficacious in an NMDA rodent model of RGC depletion and improved RGC function (nSTR) in the NMDA model measured by ERG, consistent with previous published data. Treatment of rat eyes with NMDA mimics the RGC depletion observed in Glaucoma. NMDA depletes RGC layer and impairs vision, an ideal model for investigating the effect of transplanted human Müller cells in vivo. NMDA suppresses b-wave and nSTR (negative scotopic response) as measured by electroretinogram. nSTR directly relates to RGC function. Müller cells differentiated from RC-9 cells and produced using the GMP compliant protocol of the invention partially recovered the nSTR, and indication that RGC function was improved, FIG. 12.

To conclude, Müller cells can be derived from hESCs such as RC-9 cells and cultured to provide biochemical and metabolic support in vitro and in vivo. The Müller cells of the invention are characterised according to Müller cell markers and express appropriate neurotrophins and anti-oxidants at biologically relevant levels. In addition the Müller cells of the invention show improved properties over published Müller cells. Furthermore, the Müller cells of the invention enhance RGC neurite outgrowth in vitro and enhance RGC function measured by ERG following in vivo transplant into damaged eyes.

1. An isolated human Müller cell, wherein the cell:
(a) expresses detectable levels of CD29, Vimentin, CD44 and Nestin and does not express detectable levels of Tra-1-60, and
(b) is able to secrete the neurotrophins BDNF and PEDF.

2. A purified, substantially homogenous population of two or more Müller cells according to claim 1.

3. A population of human Müller cells, wherein at least 95% percent of the cells in the population express CD29, Vimentin, CD44 and Nestin to a detectable level and less than 5% of cells express Tra-1-60 to a detectable level, and wherein the cells are able to secrete the neurotrophins BDNF and PEDF.

4. A Müller cell derived from human embryonic stem cells, wherein the cell:
(a) expresses detectable levels of CD29, Vimentin, CD44 and Nestin and does not express detectable levels of Tra-1-60, and
(b) is able to secrete the neurotrophins BDNF and PEDF.

5. The Müller cell according to claim 4, wherein the cell is derived from RC-9 human embryonic stem cells.

6. A purified, substantially homogeneous population of two or more Müller cells according to claim 4 or 5.

7. A method for producing therapeutic grade human Müller cells said method comprising,
a) culturing RC-9 human embryonic stem cells in suspension in a plate-based system in xeno- and serum-free medium in the presence of a ROCK signalling pathway inhibitor and a Wnt inhibitor for at least 15 days,
b) supplementing the xeno- and serum-free medium of step (a) with a synthetic cell adhesion promoter and culturing said cells for at least 8 days,
c) supplementing the xeno- and serum-free medium of step (b) with a synthetic enriched growth factor and an agonist of Smoothened protein of the hedgehog signalling pathway and culturing said cells for at least 3 days,
d) culturing said cells from step (c) for an additional 2-300 days supplementing the xeno- and serum-free medium of step (c) with retinoic acid until retinal organoids are visible,
e) dissociating said retinal organoids to isolate Müller cells.

8. A pharmaceutical composition comprising the Müller cell of claim 1, 4 or 5, or the population of Müller cells of claim 2, 3 or 6, or a Müller cell derivable from claim 7, and a pharmaceutically acceptable carrier.

9. A pharmaceutical composition comprising a population of Müller cells obtainable from a method comprising:
(a) culturing stem cells in suspension in a plate-based system in xeno- and serum-free medium in the presence of a ROCK signalling pathway inhibitor and a Wnt inhibitor for at least 15 days,
(b) supplementing the xeno- and serum-free medium of step (a) with a synthetic cell adhesion promoter and culturing said cells for at least 8 days,
(c) supplementing the xeno- and serum-free medium of step (b) with a synthetic enriched growth factor and an agonist of Smoothened protein of the hedgehog signalling pathway and culturing said cells for at least 3 days,
(d) culturing said cells from step (c) for an additional 2-300 days supplementing the xeno- and serum-free medium of step (c) with retinoic acid until retinal organoids are visible,
(e) dissociating said retinal organoids to isolate Müller cells; and a pharmaceutically acceptable carrier.

10. The pharmaceutical composition of claim 9, wherein the stem cells are human embryonic stem cells, optionally RC-9 human embryonic stem cells.

11. The pharmaceutical composition of any one of claims 8 to 10, wherein the Müller cells have the characteristics of the Müller cells of claim 1.

12. A method of treating a retinal disease or condition, comprising administering the pharmaceutical composition of claims 8 to 10, the Müller cell of claim 1, 4 or 5, the population of Müller cells of claim 2, 3 or 6, or a Müller cell derivable from claim 7, to a patient in need thereof.

13. A method of treating a retinal disease or condition comprising administering a pharmaceutical composition to a patient in need thereof, wherein the pharmaceutical composition comprises a pharmaceutically acceptable carrier and a population of Müller cells, wherein the Müller cells are obtained from a method comprising:
(a) culturing stem cells in suspension in a plate-based system in xeno- and serum-free medium in the presence of a ROCK signalling pathway inhibitor and a Wnt inhibitor for at least 15 days,
(b) supplementing the xeno- and serum-free medium of step (a) with a synthetic cell adhesion promoter and culturing said cells for at least 8 days,
(c) supplementing the xeno- and serum-free medium of step (b) with a synthetic enriched growth factor and an agonist of Smoothened protein of the hedgehog signalling pathway and culturing said cells for at least 3 days,
(d) culturing said cells from step (c) for an additional 2-300 days supplementing the xeno- and serum-free medium of step (c) with retinoic acid until retinal organoids are visible,
(e) dissociating said retinal organoids to isolate Müller cells.

14. The method of claim 13, wherein the stem cells are human embryonic stem cells, optionally RC-9 human embryonic stem cells.

15. The method of any one of claims 12 to 14, wherein the retinal disease or condition is vision loss, blindness, glaucoma, optic nerve damage, optic nerve degeneration, a disease that cause optic nerve damage or degeneration, dominant optic atrophy, Leber hereditary optic neuropathy, congenital amaurosis, optic neuritis, a mitochondrial disorder that causes optic nerve damage, a ganglion cell disease, an optic nerve cell disease, or ischemic optic neuropathy.

16. The method of claim 15, wherein the glaucoma is:
(a) primary glaucoma, including primary open angle glaucoma, acute primary angle closure glaucoma, chronic primary angle closure glaucoma, normal tension glaucoma, childhood glaucoma and juvenile glaucoma; or
(b) secondary glaucoma, including developmental glaucoma such as Axenfeld anomaly, Rieger anomaly, Reiger syndrome, Aniridia and Peters anomaly, traumatic glaucoma, steroid induced glaucoma, pseudoexfoliative glaucoma, pigmentary glaucoma, uveitic glaucoma, neovascular glaucoma, mixed mechanism glaucoma, irido corneal endothelial syndrome, a disease causing optic nerve injury, Posner-Schlossman syndrome, juvenile rheumatoid arthritis and Ankylosing spondylitis with secondary uveitis.

17. A method for producing therapeutic grade human Müller cells said method comprising:
a) culturing stem cells in suspension in a plate-based system in xeno- and serum-free medium in the presence of a ROCK signalling pathway inhibitor and a Wnt inhibitor for at least 15 days;
b) supplementing the xeno- and serum-free medium of step (a) with a synthetic cell adhesion promoter and culturing said cells for at least 8 days;
c) supplementing the xeno- and serum-free medium of step (b) with a synthetic enriched growth factor and an agonist of Smoothened protein of the hedgehog signalling pathway and culturing said cells for at least 3 days;
d) culturing said cells from step (c) for an additional 2-300 days supplementing the xeno- and serum-free medium of step (c) with retinoic acid until retinal organoids are visible; and
e) dissociating said retinal organoids to isolate Müller cells.

18. The method of claim 17, wherein the synthetic cell adhesion promoter in step (b) is a synthetic vitronectin-based glycoprotein.

19. The method of claim 17 or 18, wherein the synthetic enriched growth factor in step (c) is human platelet lysate.

20. The method of any one of claims 17 to 19, wherein the cell culture medium in stage (d) additionally comprises human transferrin.

21. A method for producing Müller cells said method comprising:
a) culturing retinal organoids in human platelet lysate; and
b) dissociating said retinal organoids without dissection to isolate Müller cells.

22. A method for producing a pure population of Müller cells said method comprising:
a) culturing retinal organoids;
b) dissociating said retinal organoids to isolate an enriched Müller cell suspension;
c) culturing said Müller cells on a fibronectin coated surface; and
d) isolating said Müller cells on fibronectin to form a pure population of the same.

23. A method according to claim 22, wherein a further step (e) comprises expanding said pure population of cells from step (d) in a medium supplemented with fibroblast growth factor (FGF) or epidermal growth factor (EGF) or FGF and EGF.

24. A method for producing therapeutic grade pure human Müller cells said method comprising:
a) culturing stem cells in suspension in a plate-based system in xeno- and serum-free medium in the presence of a ROCK signalling pathway inhibitor and a Wnt inhibitor for at least 15 days;
b) supplementing the xeno- and serum-free medium of step (a) with a synthetic cell adhesion promoter and culturing said cells for at least 8 days;
c) supplementing the xeno- and serum-free medium of step (b) with a synthetic enriched growth factor and an agonist of Smoothened protein of the hedgehog signalling pathway and culturing said cells for at least 3 days;
d) culturing said cells from step (c) for an additional 2-300 days supplementing the xeno- and serum-free medium of step (c) with retinoic acid until retinal organoids are visible;
e) dissociating said retinal organoids to isolate an enriched Müller cell suspension;
f) culturing said Müller cells on a fibronectin coated surface; and
g) isolating said Müller cells on fibronectin to form a pure population of the same.

25. The method according to claim 24, wherein a further step comprises expanding said pure population of cells from step (g) in a medium supplemented with fibroblast growth factor (FGF) or epidermal growth factor (EGF) or FGF and EGF.

26. The method of any preceding claim wherein the plate-based system is a V-bottomed well plate.

27. Müller cells obtainable by any one of claims 17 to 26.

28. The use of the Müller cells of claim 27 in the manufacture of a medicament for the treatment of a condition associated with cell loss or cell damage.

29. The use of the Müller cells of claim 27 in the manufacture of a medicament for the treatment of any one of age-related macular degeneration, proliferative diabetic retinopathy, proliferative vitreoretinopathy, retinal detachment, retinitis pigmentosa, glaucoma or optic nerve injury and degeneration.

30. A cell culture medium for the differentiation of Müller cells consisting essentially of a minimum essential synthetic basal medium, a carbon source, non-essential amino acids a ROCK inhibitor, human platelet lysate, Wnt antagonist and a GMP-compliant synthetic vitronectin, synthetic vitronectin-based substrate or hydrogel scaffold.

31. A cell culture medium according to claim 30 which includes an agonist of Smoothened protein of the hedgehog signalling pathway.

32. A cell culture medium for the differentiation of Müller cells consisting essentially of a minimum essential synthetic basal medium, N2 supplement, retinoic acid, and human platelet lysate.
